(19) United States
(12) Patent Application Publication
(10) Pub. No.: US 2006/0018858 A1
(43) Pub. Date: Jan. 26, 2006

(54) Skin care composition

(76) Inventors: Minghua Chen, Kobe (JP); Peiwen Sun, Chao Yang District (CN); Akira Date, Ashiya (JP); Etsuko Yuyama, Kobe (JP); Donald Lynn Bissett, Hamilton, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/187,449

(22) Filed: Jul. 22, 2005

Related U.S. Application Data
(60) Provisional application No. 60/590,561, filed on Jul. 23, 2004.

(51) Int. Cl. A61K 31/7048; A61K 8/60

(52) U.S. Cl. 424/70.13; 514/27

(57) ABSTRACT

Disclosed is a skin care composition comprising: (1) from about 0.001% to about 10% of a flavonoid compound; (2) from about 0.01% to about 15% of a vitamin B3 compound; and (3) a dermatologically acceptable oil continuous phase carrier.

### CROSS REFERENCE TO RELATED APPLICATION

This application claims the benefit of U.S. Provisional Application No. 60/590,561, filed on Jul. 23, 2004.

### FIELD OF THE INVENTION

The present invention relates to a skin care composition that provides various improved skin care benefits such as skin lightening and anti-aging. The present invention particularly relates to compositions in oil continuous phase form.

### BACKGROUND

Various treatment for the skin are proposed for delaying, minimizing or even eliminating skin hyperpigmentation (age spots, freckles, blotches, darkening, sallowness, uneven tone, and the like), wrinkling and other chronical changes typically associated with skin aging or environmental damage to human skin. Such treatments range from application of specialty cosmetics such as packs and masks, oral intake of vitamins, to chemical peeling, laser surgery, photofacial, and others. Generally, it is believed that effective treatment requires more time, physical, and financial commitment. There is a high desire for a treatment which is effective, but is safe and reasonably priced such that the consumer can use daily.

Various skin lightening agents and anti-aging agents are known in the art. It is also known that combination of actives may provide synergistic benefit. Flavonoids such as hesperidin are known in the art for use on the skin, for example in Japanese patent publications A11-346792, A2002-255827, A2003-137734, and United States Patent Application Publication 2002/13481.

For providing a skin care composition that is safe and effective for the general consumer, a combination of active agents which can be used in a variety of phase types is desired. Further, make-up compositions, such as foundations and lipsticks, providing such benefits are also desired. Most make-up compositions are made in oil-continuous phase form. It is generally known to one skilled in the art that water-continuous phase compositions are typically more effective in skin penetrating of skin active agents compared to oil-continuous phase compositions.

Based on the foregoing, there is a need for an oil continuous phase skin care composition which provides safe and effective skin care treatment benefit over a wide range of formulations. Specifically, there is a need for a composition which provides skin lightening benefit and/or anti-aging benefit.

None of the existing art provides all of the advantages and benefits of the present invention.

### SUMMARY

The present invention is directed to a skin care composition comprising:
(1) from about 0.001% to about 10% of a flavonoid compound;
(2) from about 0.01% to about 15% of a vitamin B3 compound; and
(3) a dermatologically acceptable oil continuous phase carrier.

The present invention is also directed to a method of providing skin lightening benefit comprising the steps of: applying to the skin the aforementioned composition.

The present invention is also directed to a method of providing anti-aging benefit to the skin comprising the steps of: applying to the skin the aforementioned composition.

These and other features, aspects, and advantages of the present invention will become evident to those skilled in the art from a reading of the present disclosure with the appended claims.

### DETAILED DESCRIPTION

While the specification concludes with claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

Flavonoid Compound

The present composition comprises from about 0.001% to about 10%, preferably from about 0.01% to about 5%, more preferably from about 0.05% to about 1% of a flavonoid compound. Flavonoid compounds are known to provide antioxidant, UV absorbing, and radical scavenging benefits. Flavonoid compounds are also known to be effective in strengthening collagen structure.

Flavonoid compounds useful herein are derived from either 2-phenylbenzopyrone (I) or 3-phenylbenzopyrone (II) skeleton structure as follows. (McGraw-Hill encyclopedia of Science and technology)

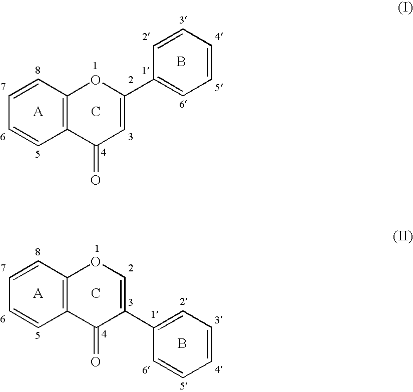

Flavonoid compounds can be further classified into different groups, depending on the oxidation level or substitution pattern of their heterocylic ring (ring C). Flavonoid compounds useful herein include unsubstituted flavanones, substituted flavanones, unsubstituted flavones, substituted flavones, unsubstituted chalcones, substituted chalcones, unsubstituted isoflavones, and substituted isoflavones. By the term “substituted” as used herein means flavonoid compounds wherein one or more hydrogen atoms of the skeleton structure as described above has been independently replaced with hydroxyl, C1-C8 alkyl, C1-C4 alkoxyl, O-glycoside, and the like or a mixture of these substituents. Flavonoid compounds particularly useful herein are selected from the group consisting of substituted flavanones, substituted flavones, substituted chalcones, substituted isoflavones, and mixtures thereof.

Flavonoid compounds can be obtained as extracts from natural sources such as plants. Examples of suitable flavonoid compounds include, but are not limited to, flavanone (unsubstituted), flavanonol (3′-hydroxy flavanone), pinocembrin (5,7-dihydroxy flavanone), pinostrobin (5-hydroxyl-7-methoxy flavanone), liquiritigenin (7,4′-dihydroxyflavanone), liquiritin (4′-glucoside-7,4′-dihydroxyflavanone), butin (7,3′,4′-trihydroxy flavanone), sakuranetin (5,4′-dihydroxy-7-methoxy flavanone), sakuranin (5-glucoside-5,4′-dihydroxy-7-methoxy flavanone), isosakuranetin (5,7-dihydroxy-4′-methoxy flavanone), poncirin (7-rhamnoglucoside-5,7-dihydroxy-4′-methoxy flavanone), naringenin (5,7,4′-trihydroxy flavanone), naringin (7-rhamnoglucoside-5,7,4′-trihydroxy flavanone), hesperitin (5,7,3′-trihydroxy-4′-methoxy flavanone), hesperidin (7-rhamnoglucoside-5,7,3′-trihydroxy-4′-methoxy flavanone), flavone (unsubstituted), chrysin (5,7-hydroxy flavone), toringin (5-glucoside-5,7-hydroxy flavone), apigenin (5,7,4′-trihydroxy flavone), apiin (7-apio-glucoside-5,7,4′-trihydroxy flavone), cosmosiin (7-glucoside-5,7,4′-trihydroxy flavone), acacetin (5,7,-dihydroxy-4′-methoxy flavone), fortunellin (7-rhamnoglucoside-5,7,-dihydroxy-4′-methoxy flavone), baicalein (5,6,7-trihydroxy flavone), baicalin (7-glucuronide-5,6,7-trihydroxy flavone), scutellarin (7-glucuronide-5,6,7,4′-tetrahydroxy flavone), diosmetin (5,7,3′-trihydroxy-4′-methoxy flavone), diosmin (7-rhamoglucoside-5,7,3′-trihydroxy-4′-methoxy flavone), galangin (3,5,7-trihydoxy flavone), quercetin (3,5,7,3′,4′-pentahydroxy flavone), quercitrin (3-rhamnoside-3,5,7,3′,4′-pentahydroxy flavone), rutin (3-rhamnoglucoside-3,5,7,3′,4′-pentahydroxy flavone), rhamnetin (3,5,3′,4′-tetrahydroxy-7-methoxy flavone), xanthorhamnin (3-rhamnoside-3,5,3′,4′-tetrahydroxy-7-methoxy flavone), myricetin (5,7,3′,4′,5′-pentahydroxy flavonol), myricitrin (3-rhamnoside-5,7,3′,4′,5′-pentahydroxy flavonol), biflavones like fukugetin; ginkgetin and bilobetin, isoflavone, chalcone, all isomers of above substituted flavones, and mixtures thereof.

The flavonoid compounds useful herein can be synthetic materials derived or modified from naturally sourced material. With these chemical modifications, the flavonoid compounds can become more applicable to skin care compositions with improved solubility or compatibility with other composition components. Preferred modified flavonoid compounds are glycosylated, alkylated or acylated from naturally sourced material.

A particularly useful group of glycoside flavonoids herein are those selected from the general structural formula (III) 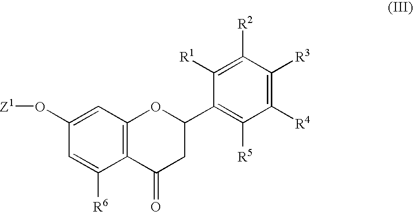
wherein R¹-R⁶ are independently selected from the group consisting of H, OH, alkoxy and hydroxyalkoxy, wherein the alkoxy or hydroxyalkoxy groups are branched or unbranched and have 1-18 carbon atoms, and wherein Z¹ is selected from the group consisting of mono- and oligoglycoside radicals. Z¹ is preferably selected from the group consisting of hexosyl radicals, more preferably rhamnosyl radicals and glucosyl radicals. It is also advantageous to use other hexosyl radicals, for example allosyl, altrosyl, galactosyl, gulosyl, idosyl, mannosyl and talosyl. It may also be advantageous according to the invention to use pentosyl radicals.

Another particularly useful group of glycoside flavonoids herein are those selected from the general structural formula (IV) 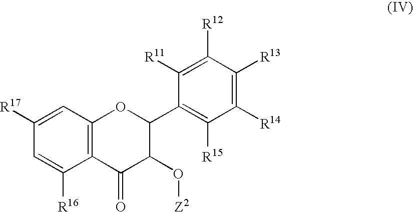
wherein R¹¹-R¹⁷ are independently selected from the group consisting of H, OH, alkoxy and hydroxyalkoxy, wherein the alkoxy or hydroxyalkoxy groups are branched or unbranched and have 1-18 carbon atoms, and wherein Z² is selected from the group consisting of mono- and oligoglycoside radicals. Z² is preferably selected from the group consisting of hexosyl radicals, more preferably rhamnosyl radicals and glucosyl radicals. It is also advantageous to use other hexosyl radicals, for example allosyl, altrosyl, galactosyl, gulosyl, idosyl, mannosyl and talosyl. It may also be advantageous according to the invention to use pentosyl radicals.

In one particularly preferred embodiment of the present invention, the glycoside flavonoid is selected from the group consisting of hesperidin, glucosyl hesperidin, rutin, glucosyl rutin, glucosyl myricitrin, glucosyl isoquercitrin, glucosyl quercitrin, methyl hesperidin, and mixtures thereof. These glucoside flavonoid compounds can be obtained by bio-chemical methods from related natural flavonoid compounds. The glucosyl group(s) can be connected to one or more hydroxides of the original substances.

A representative formula of glucosyl rutin is as follows:

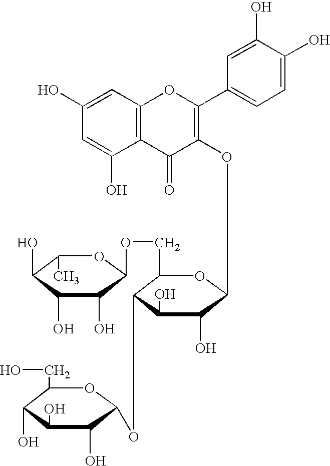

A representative formula of glucosyl hesperidin is as follows:

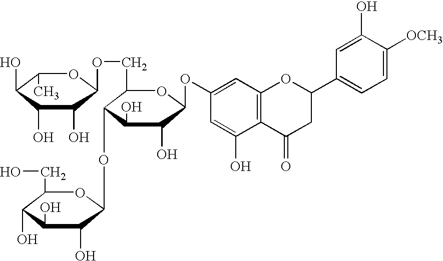

Alkylated flavonoid compounds useful herein are alkoxy or hydroxyalkoxy flavonoids that are usually derived from chemical modification of common natural flavonoids. Examples of alkylated flavonoid compounds useful herein are as follows. The formulae are merely representative, it is possible that the alkyl or hydroxyalkyl group is connected to other existing hydroxyl groups.

A representative formula of troxerutin (3′,4′,7-tri-hydroxyethoxyl-rutin) is as follows:

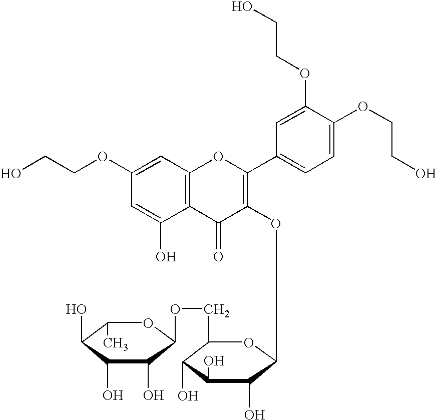

A representative formula of monoxerutin (7-hydroxyethoxyl rutin) is as follows:

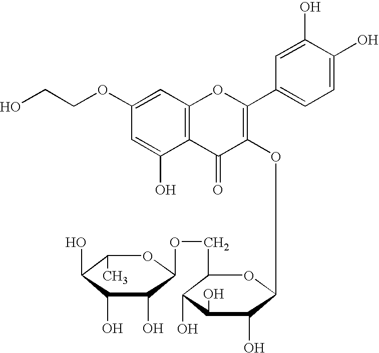

Another useful alkylated flavonoid compound, methyl hesperidin, has the general structural formula (V) 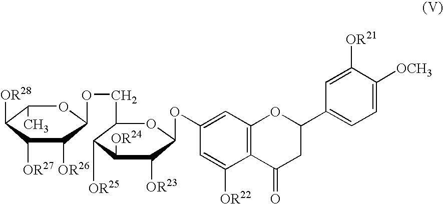
wherein R²¹-R²⁸ are independently selected from the group consisting of H or methyl, wherein at least one of R²¹-R²⁸ is methyl.

Another useful group of glycoside flavonoids herein is chalcone, which can be obtained by isomerization from any flavanone. Chalcones are highly useful in this invention due to their improved solubility which makes it easier to formulate into skin care compositions.

A representative chalcone derived from hesperidin is as follows:

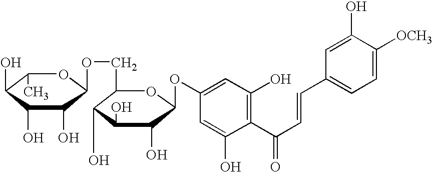

Another representative chalcone derived from hesperitin is as follows:

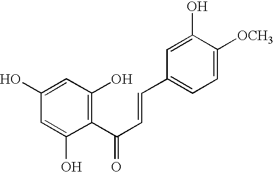

Commercially available flavonoid compounds include hesperidin, methylhesperidin, and rutin available from Alps Pharmaceutical Industry Co. Ltd. (Japan); and glucosyl hesperidin and glucosyl rutin available from Hayashibara Biochemical Laboratories, Inc. (Japan) and Toyo Sugar Refining Co. Ltd. (Japan).

Vitamin B3 Compound

The present composition comprises from about 0.01% to about 15%, preferably from about 0.1% to about 15%, more preferably from about 0.5% to about 10% of a vitamin B3 compound. Vitamin B3 compounds are known to provide, by itself, a precursor for nicotinamide adenine dinucleotide phosphate (NADP) family and its reduced form (NADPH) family of coenzymes, which enhance many metabolic enzyme reactions on the skin. Vitamin B3 compounds are also known to provide reduction in trans-epidermal water loss and excess dermal glycosaminoglycans, which are indicators for skin barrier properties.

It has been surprisingly found that, by the combined use of a flavonoid compound and a vitamin B3 compound, a composition providing synergistic skin treatment benefit over the single use of either active agent is obtained. Skin treatment benefit is particularly seen in skin lightening benefit and anti-aging benefit.

Without being bound by theory, it is believed that the flavonoid compound enhances the transportation of vitamin B3 compound. Flavonoid compounds have relatively good affinity with the cell membrane lipid bilayer, while vitamin B3 compounds have less affinity due to its generally hydrophilic structure. By the effective transportation of the 2 types of actives into the skin cells, it is believed that the 2 types of actives provide skin treatment benefits via different mechanisms in the dermis, thereby providing synergistic benefit to the skin.

Vitamin B3 compounds useful herein include, for example, those having the formula: 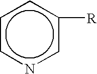
wherein R is —CONH₂⁻ (e.g., niacinamide) or —CH₂OH (e.g., nicotinyl alcohol); derivatives thereof; and salts thereof. Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Preferred vitamin B₃ compounds are niacinamide and tocopherol nicotinate, and more preferred is niacinamide. In a preferred embodiment, the vitamin B₃ compound contains a limited amount of the salt form and is more preferably substantially free of salts of a vitamin B₃ compound. Preferably the vitamin B₃ compound contains less than about 50% of such salt, and is more preferably essentially free of the salt form. Commercially available vitamin B₃ compounds that are highly useful herein include niacinamide USP available from Reilly.
Vitamin B6 Compound

The present composition preferably further comprises from about 0.001% to about 15%, preferably from about 0.01% to about 10% by weight of the composition, more preferably from about 0.01% to about 5%, of a vitamin B6 compound.

Vitamin B6 compounds are known to provide, by itself, a coenzyme for synthesis of amino acids and nucleic acids, thereby enhancing anabolic activity, such as collagen synthesis, of skin cells. Improved collagen structure is known to provide good skin tone, and lightening appearance of the skin.

It has been surprisingly found that, by the combined use of a vitamin B6 compound in addition to a flavonoid compound and a vitamin B3 compound, a composition providing significant skin treatment benefit is obtained. Skin treatment benefit is particularly seen in skin lightening benefit and anti-aging benefit.

Without being bound by theory, it is believed that the three types of actives provide an integrated effect to the skin via different mechanisms in the dermis, thereby providing synergistic benefit to the skin.

Vitamin B6 compounds useful herein include pyridoxine; esters of pyridoxine such as pyridoxine tripahnitate, pyridoxine dipalmitate, and pyridoxine dioctanoate; amines of pyridoxine such as pyridoxamine; salts of pyridoxine such as pyridoxine HCl; and derivatives thereof such as pyridoxamine, pyridoxal, pyridoxal phosphate, and pyridoxic acid. Particularly useful vitamin B6 compounds are selected from the group consisting of pyridoxine, esters of pyridoxine and salts of pyridoxine. The vitamin B6 compound can be synthetic or natural in origin and can be used as an essentially pure compound or mixtures of compounds (e.g., extracts from natural sources or mixtures of synthetic materials). As used herein, “vitamin B6” includes isomers and 6 tautomers of such. Commercially available vitamin B6 compound useful herein include, for example, pyridoxine HCl available from DSM, pyridoxine dipalmitate with tradename NIKKOL DP and pyridoxine dioctanoate with tradename NIKKOL DK available from Nikko Chemicals Co. Ltd.

Oil Continuous Phase Carrier and Composition Forms

The skin active agents of the present invention can be incorporated in various skin care compositions, including make-up compositions, for providing skin care treatment benefit such as skin lightening and anti-aging. Particularly useful composition forms and their respective suitable carriers are listed hereinbelow.

The oil continuous phase carrier may be in the form of a water-in-oil emulsion comprising, by weight of the entire composition:
(a) from about 20% to about 80% of an oil component;
(b) from about 0.1% to about 10% of a lipophilic surfactant having an HLB of less than about 8;
(c) from about 10% to about 60% of water;
(d) from 0% to about 30% of optional powder component; and
(e) from 0% to about 30% of optional thickener.
   Depending on the type and amount of optional powder component, the composition may comprise the character of a make-up composition. The water-in-oil emulsion composition herein may be in liquid or solid form. Depending on the type and amount of optional thickener, the product form may be made into a solid.

Products using this carrier type include emulsions, creams, liquid foundations, and solid foundations.

The oil continuous phase carrier may be in the form of an oil base comprising, by weight of the entire composition:
(a) from about 20% to about 80% of an oil component;
(b) from about 0.1% to about 10% of a lipophilic surfactant having an HLB of less than about 8;
(c) from 0% to about 30% of optional powder component; and
(d) from 0% to about 30% of optional thickener.
   Depending on the type and amount of optional powder component, the composition may comprise the character of a make-up composition. The oil-base composition herein may be in liquid or solid form. Depending on the type and amount of optional thickener, the product form may be made into a solid.

Products using this carrier type include liquid foundations, solid foundations, lipstick, lipcream, lipgel, mascara, and antiperspirant sticks.

Oil Component

The present composition comprises from about 20% to about 80% of an oil component forming the continuous phase the compositions herein. The oil component may be volatile or non-volatile, depending on the character of the end product.

Useful for the present invention is volatile silicone oil. The volatile silicone oil useful herein are selected from those having a boiling point of from about 60 to about 260° C., preferably those having from 2 to 7 silicon atoms. The volatile silicone oils useful herein include polyalkyl or polyaryl siloxanes with the following structure (I): 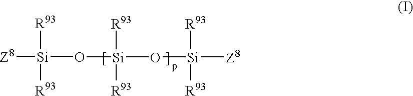
wherein R⁹³ is independently alkyl or aryl, and p is an integer from about 0 to about 5. Z⁸ represents groups which block the ends of the silicone chains. Preferably, R⁹³ groups include methyl, ethyl, propyl, phenyl, methylphenyl and phenylmethyl, Z⁸ groups include hydroxy, methyl, methoxy, ethoxy, propoxy, and aryloxy. More preferably, R⁹³ groups and Z⁸ groups are methyl groups. The preferred volatile silicone compounds are hexamethyldisiloxane, octamethyltrisiloxane, decamethyltetrasiloxane, hexadecamethylheptasiloxane. Commercially available volatile silicone compounds useful herein include octamethyltrisiloxane with tradename SH200C-1cs, decamethyltetrasiloxane with tradename SH200C-1.5cs, hexadecamethylheptasiloxane with tradename SH200C-2cs, all available from Dow Corning.

The volatile silicone oils useful herein also include a cyclic silicone compound having the formula: 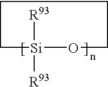
wherein R⁹³ is independently alkyl or aryl, and n is an integer of from 3 to 7.

Preferably, R⁹³ groups include methyl, ethyl, propyl, phenyl, methylphenyl and phenylmethyl. More preferably, R⁹³ groups are methyl groups. The preferred volatile silicone compounds are octamethylcyclotetrasiloxane, decamethylcyclopentasiloxane, dodecamethylcyclohexasiloxane. Commercially available volatile silicone compounds useful herein include octamethylcyclotetrasiloxane with tradename SH244, decamethylcyclopentasiloxane with tradename DC245 and SH245, and dodeamethylcyclohexasiloxane with tradename DC246; all available from Dow Corning.

Useful for the present invention is a non-volatile oil. Non-volatile oils useful herein are, for example, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, tocopherol, tocopherol acetate, avocado oil, camellia oil, turtle oil, macadamia nut oil, corn oil, mink oil, olive oil, rapeseed oil, eggyolk oil, sesame oil, persic oil, wheat germ oil, pasanqua oil, castor oil, linseed oil, safflower oil, cotton seed oil, perillic oil, soybean oil, peanut oil, tea seed oil, kaya oil, rice bran oil, china paulownia oil, Japanese paulownia oil, jojoba oil, rice germ oil, glycerol trioctanate, glycerol triisopalmiatate, trimethylolpropane triisostearate, isopropyl myristate, glycerol tri-2-ethylhexanoate, pentaerythritol tetra-2-ethylhexanoate, lanolin, liquid lanolin, liquid paraffin, squalane, vaseline, and mixtures thereof. Commercially available oils include, for example, tridecyl isononanoate with tradename Crodamol TN available from Croda, Hexalan available from Nisshin Seiyu, and tocopherol acetates available from Eisai.

Non-volatile oils useful herein also include polyalkyl or polyaryl siloxanes with the following structure (I) 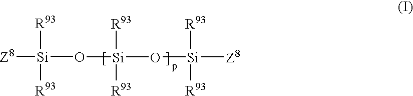
wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the skin, is compatible with the other components of the composition, and is chemically stable under normal use and storage conditions. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and SF 96 series, and from Dow Corning in their Dow Corning 200 series.

Polyalkylaryl siloxane fluids can also be used and include, for example, polymethylphenylsiloxanes. These siloxanes are available, for example, from the General Electric Company as SF 1075 methyl phenyl fluid or from Dow Corning as 556 Cosmetic Grade Fluid.

Non-volatile oils also useful herein are the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

Powder Component

The composition of the present invention may further comprise a powder component. When comprised, the powder component is included at a level of from about 0.1% to about 30% of the entire composition. The powders included in the powder component herein are typically hydrophobic in nature, or hydrophobically treated. The species and levels of the powders other than those described above are selected to provide, for example, skin feel modification, shade, coverage, UV protection benefit, good wear performance, and stability in the composition. Depending on the needs of the product, colorless/white powders may be selected for skin feel benefits, and or for providing a colorless/white foundation, and/or a make up base composition.

Powders useful for the powder component herein are clay mineral powders such as talc, mica, sericite, silica, magnesium silicate, synthetic fluorphlogopite, calcium silicate, aluminum silicate, bentonite and montmorillonite; pearl powders such as alumina, barium sulfate, calcium secondary phosphate, calcium carbonate, titanium oxide, finely divided titanium oxide, zirconium oxide, zinc oxide, hydroxy apatite, iron oxide, iron titanate, ultramarine blue, Prussian blue, chromium oxide, chromium hydroxide, cobalt oxide, cobalt titanate, titanium oxide coated mica; organic powders such as polyester, polyethylene, polystyrene, methyl methacrylate resin, cellulose, 12-nylon, 6-nylon, styrene-acrylic acid copolymers, polypropylene, vinyl chloride polymer, tetrafluoroethylene polymer, boron nitride, fish scale guanine, laked tar color dyes, and laked natural color dyes. Such powders may be treated with a hydrophobical treatment agent, including: silicone such as Methicone, Dimethicone and perfluoroalkylsilane; fatty material such as stearic acid; metal soap such as aluminium dimyristate; aluminium hydrogenated tallow glutamate, hydrogenated lecithin, lauroyl lysine, aluminium salt of perfluoroalkyl phosphate, and mixtures thereof.

Soft focus powders are also useful herein. What is meant by soft focus powder is a powder that is particularly effective in providing a soft focus effect to the composition, namely natural finish yet having good coverage for minimizing the appearance of skin troubles, when incorporated in the defined amount. Spherical soft focus powders useful herein include spherical alumina, such as those commercially available with tradename SA-Alumina Beads, available from Miyoshi Kasei Inc.

Organic oil absorbing powders are useful herein, such as silicone elastomers, and methyl methacrylate copolymers. Commercially available powders useful herein include vinyl dimethicone/methicone silsesquioxane crosspolymer with tradename KSP-100 and KSP-101 available from ShinEtsu Chemical, hardened polyorgano siloxane elastomers with tradename TREFIL E-506C available from Dow Corning, and methyl methacrylate copolymer with tradename SA-GMP-0820 available from GANZ Chemical Co., Ltd. surface treated by Miyoshi Kasei, Inc.

Spherical powders other than the soft focus powders and oil absorbing powders may also be used. Unlimited examples of materials useful for making the spherical powders are; polyacrylates, silicates, sulfates, metal dioxides, carbonates, celluloses, polyalkylenes, vinyl acetates, polystyrenes, polyamides, acrylic acid ethers, silicones, and mixtures and complexes thereof. Specifically, materials useful herein include polyacrylates such as nylon, silicates such as calcium silicate, magnesium silicate, barium silicate, aluminium silicate and silica beads; metal dioxides such as titanium dioxide and aluminium hydroxide; carbonates such as calcium carbonate, magnesium carbonate; celluloses; polyalkylenes such as polyethylene, and polypropylene; polyvinyl acetates; polystyrenes; polyamides; polyvinyl pyrrolidones; and silicones such as polyorganosilsesquioxane resin.

Commercially available spherical powders highly useful herein include Nylon-12 with tradename NYLON POWDER series available from Toray.

Lipophilic Surfactant

The composition of the present invention may comprise a lipophilic surfactant. When incorporated in water-in-oil emulsion compositions of the present invention, the amount included is preferably from about 0.1% to about 10%. When incorporated in solid water-in-oil emulsion forms, the amount included is preferably from about 1% to about 5%. Without being bound by theory, the species and levels of the lipophilic surfactant herein are believed to provide a stable water-in-oil emulsion in view of the other components of the present invention. The lipophilic surfactant herein has an HLB value of less than about 8.

The HLB value is a theoretical index value which describes the hydrophilicity-hydrophobicity balance of a specific compound. Generally, it is recognized that the HLB index ranges from 0 (very hydrophobic) to 40 (very hydrophilic). The HLB value of the lipophilic surfactants may be found in tables and charts known in the art, or may be calculated with the following general equation: HLB=7+(hydrophobic group values)+(hydrophilic group values). The HLB and methods for calculating the HLB of a compound are explained in detail in Surfactant Science Series, Vol. 1: Nonionic Surfactants”, pp 606-13, M. J. Schick (Marcel Dekker Inc., New York, 1966).

The lipophilic surfactant can be an ester-type surfactant. Ester-type surfactants useful herein include: sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

Commercially available ester-type surfactants are, for example, sorbitan isostearate having a tradename Crill 6 available from Croda, and sorbitan sesquioleate with tradename Arlacel 83 available from Kao Atras.

The lipophilic surfactant can be a silicone-type surfactant. Silicone-type surfactants useful herein are (i), (ii), and (iii) as shown below, and mixtures thereof.
(i) dimethicone copolyols having the formulation: 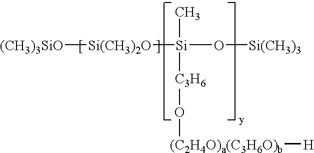
   wherein x is an integer from 5 to 100, y is an integer from 1 to 50, a is zero or greater, b is zero or greater, the average sum of a+b being 1-100.
(ii) dimethicone copolyols having the formulation: 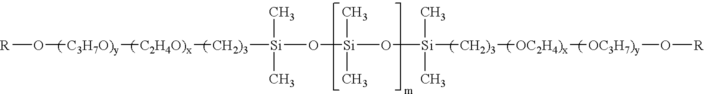
   wherein R is selected from the group consisting of hydrogen, methyl, and combinations thereof, m is an integer from 5 to 100, x is independently zero or greater, y is independently zero or greater, the sum of x+y being 1-100.
(iii) branched polyether-polydiorganosiloxane emulsifiers herein having the formulation: 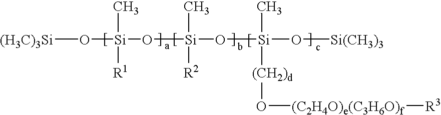
   wherein R¹ is an alkyl group having from about 1 to about 20 carbons; R² is 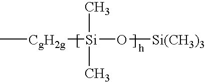
   wherein g is from about 1 to about 5, and h is from about 5 to about 20; R³ is H or an alkyl group having from about 1 to about 5 carbons; e is from about 5 to about 20; f is from about 0 to about 10; a is from about 20 to about 100; b is from about 1 to about 15; c is from about 1 to about 15; and d is from about 1 to about 5.

Commercially available silicone-type surfactants are, for example, dimethicone copolyols DC5225C, BY22-012, BY22-008, SH3746M, SH3771M, SH3772M, SH3773M, SH3775M, SH3748, SH3749, and DC5200, all available from Dow Corning, and branched polyether-polydiorganosiloxane emulsifiers such as PEG-9 polydimethylsiloxyethyl Dimethicone, having an HLB of about 4 and a molecular weight of about 6,000 having a tradename KF 6028 available from ShinEtsu Chemical.

In a preferred embodiment, the lipophilic surfactant is a mixture of at least one ester-type surfactant and at least one silicone-type surfactant to provide a stable emulsion for the other essential components of the present invention.

Water

The water-in-oil emulsion compositions of the present invention comprise water in an amount sufficient to provide a discontinuous aqueous phase. More preferably, the solid form compositions of the present invention comprise from about 10% to about 40% of water, and the liquid form compositions comprise from about 10% to about 60% of water.

In the present invention, deionized water is typically used. Water from natural sources including mineral cations can also be used, depending on the desired characteristic of the product.

Thickener

The present composition may comprise a thickener. Thickeners can be used for adding viscosity to liquid form compositions, and for solidifying solid form compositions. When comprised, the thickener is included at a level of from about 0.1% to about 30% of the entire composition. Solid forms comprise thickeners, typically solid wax, for solidifying the composition.

The thickeners useful herein are selected from the group consisting of fatty compounds, solid wax, gelling agents, inorganic thickeners, silicone elastomers, and mixtures thereof. The amount and type of thickeners are selected according to the desired viscosity and characteristics of the product.

Fatty Compounds

Fatty compounds useful herein include stearic acid, palmitic acid, stearyl alcohol, cetyl alcohol, behenyl alcohol, stearic acid, palmitic acid, the polyethylene glycol ether of stearyl alcohol or cetyl alcohol having an average of about 1 to about 5 ethylene oxide units, and mixtures thereof. Preferred fatty compounds are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof.

Solid Wax

The composition of the present invention may comprise a solid wax. The solid compositions of the present invention preferably comprise, by weight of the entire composition, from about 1% to about 20% of solid wax. Without being bound by theory, the species and levels of the solid wax herein is believed to provide consistency to the composition and coverage to the skin, while not negatively contributing to the spreadability upon application to the skin, and fresh and light feel of the skin.

The solid waxes useful herein are paraffin wax, microcrystalline wax, ozokerite wax, ceresin wax, carnauba wax, candellila wax, eicosanyl behenate, and mixtures thereof. A mixture of waxes is preferably used.

Commercially available solid waxes useful herein include: Candelilla wax NC-1630 available from Cerarica Noda, Ozokerite wax SP-1021 available from Strahl & Pitsh, and Eicosanyl behenate available from Cas Chemical.

Gelling Agents

The gelling agents useful as thickeners of the present invention include esters and amides of fatty acid gellants, hydroxy acids, hydroxy fatty acids, other amide gellants, and crystalline gellants.

N-acyl amino acid amides useful herein are prepared from glutamic acid, lysine, glutamine, aspartic acid and mixtures thereof. Particularly preferred are n-acyl glutamic acid amides corresponding to the following formula:
R²—NH—CO—(CH₂)₂—CH—(NH—CO—R¹)—CO—NH—R²
wherein R¹ is an aliphatic hydrocarbon radical having from about 12 to about 22 carbon atoms, and R² is an aliphatic hydrocarbon radical having from about 4 to about 12 carbon atoms. Non-limiting examples of these include n-lauroyl-L-glutamic acid dibutyl amide, n-stearoyl-L-glutamic acid diheptyl amide, and mixtures thereof. Most preferred is n-lauroyl-L-glutamic acid dibutyl amide, also referred to as dibutyl lauroyl glutamide. This material is commercially available with tradename Gelling agent GP-1 available from Ajinomoto.

Other gelling agents suitable for use in the compositions include 12-hydroxystearic acid, esters of 12-hydroxystearic acid, amides of 12-hydroxystearic acid and combinations thereof. These preferred gellants include those which correspond to the following formula:
R¹—CO—(CH₂)₁₀—CH—(OH)—(CH₂)₅—CH₃
wherein R¹ is R² or NR²R³; and R² and R³ are hydrogen, or an alkyl, aryl, or arylalkyl radical which is branched linear or cyclic and has from about 1 to about 22 carbon atoms; preferably, from about 1 to about 18 carbon atoms. R² and R³ may be either the same or different; however, at least one is preferably a hydrogen atom. Preferred among these gellants are those selected from the group consisting of 12-hydroxystearic acid, 12-hydroxystearic acid methyl ester, 12-hydroxystearic acid ethyl ester, 12-hydroxystearic acid stearyl ester, 12-hydroxystearic acid benzyl ester, 12-hydroxystearic acid amide, isopropyl amide of 12-hydroxystearic acid, butyl amide of 12-hydroxystearic acid, benzyl amide of 12-hydroxystearic acid, phenyl amide of 12-hydroxystearic acid, t-butyl amide of 12-hydroxystearic acid, cyclohexyl amide of 12-hydroxystearic acid, 1-adamantyl amide of 12-hydroxystearic acid, 2-adamantyl amide of 12-hydroxystearic acid, diisopropyl amide of 12-hydroxystearic acid, and mixtures thereof; even more preferably, 12-hydroxystearic acid, isopropyl amide of 12-hydroxystearic acid, and combinations thereof. Most preferred is 12-hydroxystearic acid.

Suitable amide gellants include disubstituted or branched monoamide gellants, monosubstituted or branched diamide gellants, triamide gellants, and combinations thereof, excluding the n-acyl amino acid derivatives selected from the group consisting of n-acyl amino acid amides, n-acyl amino acid esters prepared from glutamic acid, lysine, glutamine, apartic acid, and combinations thereof, and which are specifically disclosed in U.S. Pat. No. 5,429,816.

Alkyl amides or di- and tri-basic carboxylic acids or anhydrides suitable for use in the composition include alkyl amides of citric acid, tricarballylic acid, aconitic acid, nitrilotriacetic acid, succinic acid and itaconic acid such as 1,2,3-propane tributylamide, 2-hydroxy-1,2,3-propane tributylamide, 1-propene-1,2,3-triotylamide, N,N′,N″-tri(acetodecylamide)amine, 2-dodecyl-N,N′-dihexylsuccinamide, and 2 dodecyl-N,N′-dibutylsuccinamide. Preferred are alkyl amides of di-carboxylic acids such as di-amides of alkyl succinic acids, alkenyl succinic acids, alkyl succinic anhydrides and alkenyl succinic anhydrides, more preferably 2-dodecyl-N,N′-dibutylsuccinamide.

Inorganic Thickeners

Inorganic thickeners useful herein include hectorite, bentonite, montmorillonite, and bentone clays which have been modified to be compatible with oil. Preferably, the modification is quaternization with an ammonium compound. Preferable inorganic thickeners include quaternary ammonium modified hectorite. Commercially available oil swelling clay materials include benzyldimethyl stearyl ammonium hectorite with tradename Bentone 38 available from Elementis.

Silicone Elastomers

Suitable for use herein are silicone elastomers which can be emulsifying or non-emulsifying crosslinked siloxane elastomers or mixtures thereof. The term “non-emulsifying,” as used herein, defines crosslinked organopolysiloxane elastomers from which polyoxyalkylene units are absent. The term “emulsifying,” as used herein, means crosslinked organopolysiloxane elastomers having at least one polyoxyalkylene (e.g., polyoxyethylene or polyoxypropylene) unit. Non-emulsifying elastomers useful in the present invention are formed via crosslinking organohydroenpolysiloxanes with an alpha, omega-diene. Emulsifying elastomers herein include polyoxyalkylene modified elastomers formed via crosslinking from organohydrogenpolysiloxanes with polyoxyalkylene dienes or organohydrogenpolysiloxanes containing at least one polyether group crosslinked with an alpha, omega-diene. Emulsifying crosslinked organopolysiloxane elastomer can notably be chosen from the crosslinked polymers described in U.S. Pat. Nos. 5,412,004, 5,837,793, and 5,811,487. In addition, an emulsifying elastomer comprised of dimethicone copolyol crosspolymer (and dimethicone) is available from Shin Etsu under the tradename KSG-21.

Non-emulsifying elastomers are dimethicone/vinyl dimethicone crosspolymers. Such dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers useful in the present invention and processes for making them are further described in U.S. Pat. Nos. 4,970,252, 5,760,116, and 5,654,362. Additional crosslinked organopolysiloxane elastomers useful in the present invention are disclosed in Japanese Patent Application JP 61-18708, assigned to Pola Kasei Kogyo KK. Commercially available elastomers preferred for use herein are Dow Corning's 9040 silicone elastomer blend, and Shin Etsu's KSG-21.

Humectant

The composition of the present invention may further comprise a humectant. When incorporated in water-in-oil emulsions, preferably the amount is from about 1% to about 15%, more preferably from about 2% to about 7%.

The humectants herein are selected from the group consisting of polyhydric alcohols, water soluble alkoxylated nonionic polymers, and mixtures thereof. Polyhydric alcohols useful herein include glycerin, propylene glycol, 1,3-butylene glycol, dipropylene glycol, diglycerin, sodium hyaluronate, and mixtures thereof.

Commercially available humectants herein include: glycerin available from Asahi Denka; propylene glycol with tradename LEXOL PG-865/855 available from Inolex, 1,2-PROPYLENE GLYCOL USP available from BASF; 1,3-butylene glycol available from Kyowa Hakko Kogyo; dipropylene glycol with the same tradename available from BASF; diglycerin with tradename DIGLYCEROL available from Solvay GmbH; sodium hyaluronate with tradenames ACTIMOIST available from Active Organics, AVIAN SODIUM HYALURONATE series available from Intergen, HYALURONIC ACID Na available from Ichimaru Pharcos.

Film Forming Polymer

The compositions of the present invention may further comprise a film forming polymer, for imparting wear and/or transfer resistant properties. When included, such materials are typically used in an amount of from about 0.5% to about 20%, preferably from about 0.5% to about 10% by weight, more preferably from about 1% to about 8%, by weight of the composition. Preferred polymers form a non-tacky film which is removable with water used with cleansers such as soap.

Examples of suitable film forming polymeric materials include:
a) sulfopolyester resins, such as AQ sulfopolyester resins, such as AQ29D, AQ35S, AQ38D, AQ38S, AQ48S, and AQ55S (available from Eastman Chemicals);
b) polyvinylacetate/polyvinyl alcohol polymers, such as Vinex resins available from Air Products, including Vinex 2034, Vinex 2144, and Vinex 2019;
c) acrylic resins, including water dispersible acrylic resins available from National Starch under the trade name “Dermacryl”, including Dermacryl LT;
d) polyvinylpyrrolidones (PVP), including Luviskol K17, K30 and K90 (available from BASF), water soluble copolymers of PVP, including PVP/VA S-630 and W-735 and PVP/dimethylaminoethylmethacrylate Copolymers such as Copolymer 845 and Copolymer 937 available from ISP, as well as other PVP polymers disclosed by E. S. Barabas in the Encyclopedia of Polymer Science and Engineering, 2 Ed. Vol. 17 pp. 198-257;
e) high molecular weight silicones such as dimethicone and organic-substituted dimethicones, especially those with viscosities of greater than about 50,000 mPas;
f) high molecular weight hydrocarbon polymers with viscosities of greater than about 50,000 mPas;
g) organosiloxanes, including organosiloxane resins, fluid diorganopolysiloxane polymers and silicone ester waxes.

Examples of these polymers and cosmetic compositions containing them are found in PCT publication Nos. WO96/33689, published Oct. 31, 1996; WO97/17058, published May 15, 1997; and U.S. Pat. No. 5,505,937 issued to Castrogiovanni et al. Apr. 9, 1996, all incorporated herein by reference. Additional film forming polymers suitable for use herein include the water-insoluble polymer materials in aqueous emulsion and water soluble film forming polymers described in PCT publication No. WO98/18431, published May 7, 1998, incorporated herein by reference. Examples of high molecular weight hydrocarbon polymers with viscosities of greater than about 50,000 mPas include polybutene, polybutene terephthalate, polydecene, polycyclopentadiene, and similar linear and branched high molecular weight hydrocarbons.

Preferred film forming polymers include organosiloxane resins comprising combinations of R₃SiO_{1/2} “M” units, R₂SiO “D” units, RSiO_{3/2} “T” units, SiO₂ “Q” units in ratios to each other that satisfy the relationship RₙSiO_{(4-n)/2} where n is a value between 1.0 and 1.50 and R is a methyl group. Note that a small amount, up to 5%, of silanol or alkoxy functionality may also be present in the resin structure as a result of processing. The organosiloxane resins must be solid at about 25° C. and have a molecular weight range of from about 1,000 to about 10,000 grams/mole. The resin is soluble in organic solvents such as toluene, xylene, isoparaffins, and cyclosiloxanes or the volatile carrier, indicating that the resin is not sufficiently crosslinked such that the resin is insoluble in the volatile carrier. Particularly preferred are resins comprising repeating monofunctional or R₃SiO_{1/2} “M” units and the quadrofunctional or SiO₂ “Q” units, otherwise known as “MQ” resins as disclosed in U.S. Pat. No. 5,330,747, Krzysik, issued Jul. 19, 1994, incorporated herein by reference. In the present invention the ratio of the “M” to “Q” functional units is preferably about 0.7 and the value of n is 1.2. Organosiloxane resins such as these are commercially available such as Wacker 803 and 804 available from Wacker Silicones Corporation of Adrian Mich., KP545 from Shin-Etsu Chemical and G. E. 1170-002 from the General Electric Company.

Additional Skin Active Agent

The compositions of the present invention may further comprise a safe and effective amount of an additional skin active agent. The skin active agents useful herein include skin lightening agents, anti-acne agents, emollients, non-steroidal anti-inflammatory agents, topical anaesthetics, artificial tanning agents, antiseptics, anti-microbial and anti-fungal actives, skin soothing agents, sunscreening agents, skin-barrier repair agents, anti-wrinkle agents, anti-skin atrophy actives, lipids, sebum inhibitors, sebum inhibitors, skin sensates, protease inhibitors, skin tightening agents, anti-itch agents, hair growth inhibitors, desquamation enzyme enhancers, anti-glycation agents, and mixtures thereof. When included, the present composition comprises from about 0.001% to about 30%, preferably from about 0.001% to about 10% of an additional skin active agent.

The type and amount of skin active agents are selected so that the inclusion of a specific agent does not affect the stability of the composition.

Skin lightening agents useful herein refer to active ingredients that improve hyperpigmentation as compared to pre-treatment. Useful skin lightening agents herein include ascorbic acid compounds, azelaic acid, butyl hydroxyanisole, gallic acid and its derivatives, glycyrrhizinic acid, hydroquinone, kojic acid, arbutin, mulberry extract, and mixtures thereof. Use of combinations of skin lightening agents is believed to be advantageous in that they may provide skin lightening benefit through different mechanisms.

Ascorbic acid compounds useful herein include, ascorbic acid per se in the L-form, ascorbic acid salt, and derivatives thereof. Ascorbic acid salts useful herein include, sodium, potassium, lithium, calcium, magnesium, barium, ammonium and protamine salts. Ascorbic acid derivatives useful herein include, for example, esters of ascorbic acid, and ester salts of ascorbic acid. Particularly preferred ascorbic acid compounds include 2-o-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include magnesium ascorbyl phosphate available from Showa Denko, 2-o-D-glucopyranosyl-L-ascorbic acid available from Hayashibara and sodium L-ascorbyl phosphate with tradename STAY C50 available from DSM.

Other hydrophobic skin lightening agents useful herein include ascorbic acid derivatives such as ascorbyl tetraisopalmitate (for example, VC-IP available from Nikko Chemical), ascorbyl palmitate (for example available from DSM), ascorbyl dipalmitate (for example, NIKKOL CP available from Nikko Chemical); undecylenoyl phenyl alanine (for example, SEPIWHITE MSH available from Seppic); octadecenedioic acid (for example, ARLATONE DIOIC DCA available from Uniquema); oenothera biennis sead extract, and pyrus malus (apple) fruit extract, and mixtures thereof.

Other skin active agents useful herein include those selected from the group consisting of panthenol, benzoyl peroxide, 3-hydroxy benzoic acid, farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol and its esters (e.g., tocopheryl acetate), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof.

UV Absorbing Agent

The compositions of the present invention may further comprise a safe and effective amount of a UV absorbing agent. A wide variety of conventional UV protecting agents are suitable for use herein, such as those decribed in U.S. Pat. No. 5,087,445, Haffey et al, issued Feb. 11, 1992; U.S. Pat. No. 5,073,372, Turner et al, issued Dec. 17, 1991; U.S. Pat. No. 5,073,371, Turner et al., issued Dec. 17, 1991; and Segarin, et al, at Chapter VIII, pages 189 et seq., of Cosmetics Science and Technology (1972). When included, the present composition comprises from about 0.5% to about 20%, preferably from about 1% to about 15% of a UV absorbing agent.

UV absorbing agents useful herein are, for example, 2-ethylhexyl-p-methoxycinnamate (commercially available as PARSOL MCX), butylmethoxydibenzoylmethane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, Eusolex™ 6300, Octocrylene, Avobenzone (commercially available as Parsol 1789), and mixtures thereof.

Additional Components

The compositions hereof may further contain additional components such as are conventionally used in topical products, e.g., for providing aesthetic or functional benefit to the composition or skin, such as sensory benefits relating to appearance, smell, or feel, therapeutic benefits, or prophylactic benefits (it is to be understood that the above-described required materials may themselves provide such benefits).

Examples of suitable topical ingredient classes include: anti-cellulite agents, antioxidants, radical scavengers, chelating agents, vitamins and derivatives thereof, abrasives, other oil absorbents, astringents, dyes, essential oils, fragrance, structuring agents, emulsifiers, solubilizing agents, anti-caking agents, antifoaming agents, binders, buffering agents, bulking agents, denaturants, pH adjusters, propellants, reducing agents, sequestrants, cosmetic biocides, and preservatives.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### Examples 1-8

Examples 1-4 are useful as solid foundation products. Example 5 is useful as a make up base product. Example 6 is useful as a liquid foundation product. Examples 7-8 are useful as skin care cream.

Compositions

| Components | Ex. 1 | Ex. 2 | Ex. 3 | Ex. 4 |
|---|---|---|---|---|
| Cyclopentasiloxane *1 | 24 | 29 | 16 | 30 |
| PEG-9 Polydimethylsiloxyethyl Dimethicone | 1 | 1 | 1 | 1 |
| *2 | | | | |
| Tocopheryl Acetate *14 | 0.5 | 0.5 | 0.5 | 0.5 |
| Isotridecyl Isononanoate *15 | 1 | 1 | 1 | 1 |
| Sorbitan Monoisostearate *16 | 1.5 | 1.5 | 1.5 | 1.5 |
| Slurry of Iron Oxide and Cyclopentasiloxane | 2 | 2 | 2 | 2 |
| and Dimethicone and Disodium Hydro- | | | | |
| genated Glutamate *17 | | | | |
| Slurry of Titanium Dioxide and | 10 | | 10 | |
| Cyclopentasiloxane and Dimethicone and | | | | |
| Disodium Hydrogenated Glutamate *18 | | | | |
| Slurry of Titanium Dioxide and | 6 | | 6 | |
| Cyclopentasiloxane and Dimethicone and | | | | |
| Methicone *19 | | | | |
| Titanium Dioxide and Methicone *20 | | 8 | | 6 |
| Spherical Silica and Methicone *21 | 2.3 | 5 | 4.4 | 3.5 |
| Mica and Zinc Oxide and Methicone and | 3 | 6 | 7.5 | 3.5 |
| Hydroxyapatite *22 | | | | |
| Mica and Methicone *23 | 2 | 1.3 | 1 | 1 |
| Niacinamide *24 | 2 | 2 | 2 | 2 |
| Glucosyl Hesperidin *25 | 0.5 | 0.5 | 0.5 | 0.5 |
| Pyridoxine Dipalmitate *26 | 0.2 | 0.2 | 0.2 | 0.2 |
| Preservative | 0.45 | 0.45 | 0.45 | 0.45 |
| Panthenol *28 | 0.25 | 0.25 | 0.25 | 0.25 |
| Butylene Glycol *30 | 5 | 5 | 5 | 5 |
| Candelilla Wax *31 | 2 | 2 | 2 | 2 |
| Ceresin *32 | 1.8 | 1.8 | 1.8 | 1.8 |
| Water | Make to total 100 | | | |
| Components | Ex. 5 | Ex. 6 | Ex. 7 | Ex. 8 |
| Cyclopentasiloxane *1 | 38 | 24 | 18.5 | 23.5 |
| PEG-9 Polydimethylsiloxyethyl Dimethicone | 1 | 1 | | |
| *2 | | | | |
| Dimethicone and Dimethicone/Vinyl | | 3 | | |
| Dimethicone Crosspolymer *3 | | | | |
| Ethylene/acrylic acid copolymer *4 | | | 2.5 | |
| Titanium dioxide/silicone dispersion *5 | | | 0.72 | |
| Sucrose Polycottonseedate *6 | | | 0.5 | |
| Stearyl Dimethicone *7 | | | 0.5 | |
| Cyclopentasiloxane and Dimethicone | | | 8.4 | 13.5 |
| Crosspolymer *8 | | | | |
| Polymethylsilsesquioxane *9 | | | | 7.5 |
| Dimethicone Copolyol Crosspolymer *10 | | | 2.5 | 2.5 |
| Bis-PEG/PPG-14/14 Dimethicone *11 | | | 0.3 | 0.45 |
| PEG-10 Dimethicone *12 | | | 0.25 | 0.375 |
| Cetyl Ricinoleate *13 | | | 0.25 | 0.25 |
| Tocopheryl Acetate *14 | 0.5 | 0.5 | 0.5 | 0.5 |
| Isotridecyl Isononanoate *15 | 1 | 2 | 1 | 1 |
| Sorbitan Monoisostearate *16 | 1.5 | 1.5 | 1 | 1 |
| Slurry of Iron Oxide and Cyclopentasiloxane | | 2 | | |
| and Dimethicone and Disodium Hydro- | | | | |
| genated Glutamate *17 | | | | |
| Slurry of Titanium Dioxide and | | 8 | | |
| Cyclopentasiloxane and Dimethicone and | | | | |
| Disodium Hydrogenated Glutamate *18 | | | | |
| Slurry of Titanium Dioxide and | 6 | 6 | | |
| Cyclopentasiloxane and Dimethicone and | | | | |
| Methicone *19 | | | | |
| Spherical Silica and Methicone *21 | 3 | 2 | | |
| Mica and Zinc Oxide and Methicone and | 5 | 2 | | |
| Hydroxyapatite *22 | | | | |
| Niacinamide *24 | 2 | 2 | 3.5 | 4 |
| Glucosyl Hesperidin *25 | 0.5 | 0.5 | 0.5 | 0.5 |
| Pyridoxine Dipalmitate *26 | 0.2 | 0.2 | 0.2 | 0.2 |
| Promatrixyl *27 | | | 0.35 | 0.35 |
| Preservative | 0.45 | 0.45 | 0.45 | 0.45 |
| Panthenol *28 | 0.25 | 0.25 | 1 | 1 |
| Glycerin *29 | | 5 | 10 | 10 |
| Butylene Glycol *30 | 5 | | 2 | 2 |
| Candelilla Wax *31 | 4 | | | |

| | |
|---|---|
| Water | Make to total 100 |

Definitions of Components
*1 Cyclopentasiloxane SH245 available from Dow Corning
*2 PEG-9 Polydimethylsiloxyethyl Dimethicone: KF-6028 available from Shinetsu Silicone
*3 Dimethicone and Dimethicone/Vinyl Dimethicone Crosspolymer: KSG-16 available from Shinetsu Silicone
*4 Ethylene/acrylic acid copolymer: EA-209 available from Kobo Products Inc.
*5 Titanium dioxide/silicone dispersion: 70% TiO2 SAT-CR-50 Dispersion available from Kobo Products Inc.
*6 Sucrose Polycottonseedate: SEFA Cottonate available from Kobo Products Inc.
*7 Stearyl Dimethicone: DC 2503 available from Dow Corning
*8 Cyclopentasiloxane and Dimethicone Crosspolymer: DC9040 Silicone Elastomer Blend available from Dow Corning
*9 Polymethylsilsesquioxane Tospearl 2000 available from GE Toshiba Silicone
*10 Dimethicone Copolyol Crosspolymer: KSG-21 available from Shinetsu Silicone
*11 Bis-PEG/PPG-14/14 Dimethicone: Abil EM-97 available from Goldschmidt
*12 PEG-10 Dimethicone: KF-6017 available from Shinetsu Silicone
*13 Cetyl Ricinoleate: Nature Chem CR available from CasChem
*14 Tocopheryl Acetate: DL-a-Tocopheryl Acetate available from Eisai
*15 Isotridecyl Isononanoate: Crodamol TN available from Croda
*16 Sorbitan Monoisostearate: Crill 6 available from Croda
*17 Slurry of Iron Oxide and Cyclopentasiloxane and Dimethicone and Disodium Hydrogenated Glutamate: SA/NAI-Y-10/D5 (70%), SA/NAI-R-10/D5 (65%) and SA/NAI-B-10/D5 (75%) available from Miyoshi Kasei
*18 Slurry of Titanium Dioxide and Cyclopentasiloxane and Dimethicone and Disodium Hydrogenated Glutamate: SA/NAI-TR-10/D5 (80%) available from Miyoshi Kasei
* 19 Slurry of Titanium Dioxide and Cyclopentasiloxane and Dimethicone and Methicone: SAS-TTO-S-3/D5 (50%) available from Miyoshi Kasei
*20 Titanium Dioxide and Methicone: SI Titanium Dioxide IS available from Miyoshi Kasei
*21 Spherical Silica and Methicone: SI-SILDEX H-52 available from Asahi Glass Company Co., Ltd. and surface treated by Miyoshi Kasei, having an oil absorbency of more than 200 m [symbol] /100 g
*22 Mica and Zinc Oxide and Methicone and Hydroxyapatite: SI-PLV-20 available from Miyoshi Kasei
*23 Mica and Methicone: SI Mica available from Miyoshi Kasei
*24 Niacinamide: Niacinamide available from Reilly Industries Inc.
*25 Glucosyl Hesperidin: available from Alps Pharmaceutical Industry Co
*26 Pyridoxine Dipalmitate: Nikkol DP, available from Nikko Chemicals.
*27 Promatrixyl: mixture contains Palmitoyl Pentapeptide-3, Available Sederma, Inc.
*28 Panthenol: DL-Panthenol available from Alps Pharmaceutical Ind.
*29 Glycerin: Glycerin USP available from Asahi Denka
*30 Butylene Glycol: 1,3 Butylene Glycol available from Kyowa Hakko Kogyo
*31 Candelilla Wax: Candelilla wax NC-1630 available from Cerarica Noda
*32 Ceresin: Ozokerite wax SP-1021 available from Strahl & Pitsh

Method of Preparation

Examples 1-8 are prepared as follows:
1) All oil components, including silicone oil base mixtures, oil soluble solid preservatives are mixed with suitable mixer until homogeneous to make a lipophilic mixture.
2) All insoluble powder Components, as included, are mixed with suitable mixer until homogeneous to make a powder mixture. The powder mixture is pulverized using pulverizer. The powder mixture is added into the lipophilic mixture with suitable mixer until homogeneous.
3) All water soluble components are dissolved with suitable mixer until all components are completely dissolved to make a water phase. The water phase is added into the product of step 2) to effect emulsion at room temperature using homogenizer.
4) Wax components, Candelilla Wax and Ceresin, if included, are added into the product of step 3). For Examples 1 through 5, this is heated to dissolve at 80-85° C. in a sealed tank to melt the waxes.
5) Finally, for examples 1-5, the obtained emulsion is filled in an container and allowed to cool to room temperature using a cooling unit to form solid emulsion. For examples 6-8, fill the emulsion into suitable package.

### Examples 9-13

Examples 9-11 are useful as lipstick products. Example 12 is useful as a stick foundation product. Example 13 is useful as a powder foundation product.

| NO. | Component | Ex. 9 | Ex. 10 | Ex. 11 | Ex. 12 | Ex. 13 |
|---|---|---|---|---|---|---|
| 1 | Quaternium-18 Hectorite *1 | 0.2 | 0.2 | 0.2 | 0.2 | 0.3 |
| 2 | Cholesteryl Hydroxystearate *2 | 3 | 6 | 3 | 5 | 5 |
| 4 | Phenyl Trimethicone *3 | 5 | | | 5 | 7 |
| 5 | Liquid Petrolatum *4 | | 2 | 5 | | |
| 6 | Pyridoxine Dipalmitate *5 | 0.2 | 0.1 | | 0.5 | 0.2 |
| 7 | Glucosyl Hesperidin *6 | 1 | 1 | 1 | | |
| 8 | Glucosyl Rutin *7 | | | | 3 | 3 |
| 9 | Niacinamide *8 | 2 | 2 | 2 | 2 | 3.5 |
| 10 | Urea *9 | | 2 | | | |
| 11 | Glycerin *10 | 4 | 3 | 5 | 6 | 7 |
| 12 | Ozokerite *11 | 4 | 4 | 4 | 4 | 4 |
| 13 | Microcrystalline Wax *12 | 1.5 | 1.5 | 1.5 | 1.5 | 1.5 |
| 14 | Candelilla Wax *13 | 5 | 5 | 5 | 5 | 5 |
| 15 | Paraffin *14 | 3 | 3 | 3 | 3 | 2 |
| 16 | Diglyceryl | 20.6 | 22.2 | 22.3 | 20.8 | |
| | Sebacate/Isopalmitate *15 | | | | | |
| 17 | Absorption Refined Lanolin *16 | 2.5 | 2.5 | 2.5 | 2.5 | 2.5 |
| 18 | Lanolin Oil *17 | 20 | 20 | 20 | | |
| 19 | Trioctanoin *18 | 7 | 7 | 7 | 16 | 18.5 |
| 20 | Isotridecyl Isononanoate *19 | 3 | 3 | 3 | 5 | 10 |
| 21 | Ethylhexyl Methoxycinnamate | 3.5 | | | | |
| 22 | Preservatives | 0.5 | 0.5 | 0.5 | 0.5 | 0.5 |
| 23 | Powder Pigments | 14 | 15 | 15 | 20 | 30 |

Definitions of Components
*1 Quaternium-18 Hectorite: Bentone 38 available from Rheox Inc.
*2 Cholesteryl Hydroxystearate: Salacos HS available from Nisshin Oil Mills, Ltd.
*3 Phenyl Trimethicone: Silicone Oil KF-56 available from Shinetsu Silicone
*4 Liquid Petrolatum: Liquid Petrolatum available from Witco Chemical
*5 Pyridoxine Dipalmitate: Nikkol DP, available from Nikko Chemicals
*6 Glucosyl Hesperidin: available from Alps Pharmaceutical Industry Co
*7 Glucosyl Rutin: alpha-GRutin, available from Toyo Sugar Refining
*8 Niacinamide: Niacinamide available from DSM
*9 Urea: Urea available from Taisei Chemical
*10 Glycerin: Glycerin USP available from Asahi Denka
*11 Ozokerite: Ozokerite wax SP-1021 available from Strahl & Pitsh
*12 Microcrystalline Wax: Multiwax 180-M Yellow available from Witco Chemical
*13 Candelilla Wax: Candelilla wax NC-1630 available from Noda wax
*14 Paraffin: Paraffin wax FT-150 available from Sazole
*15 Diglyceryl Sebacate/Isopalmitale: Salacos DGS-16 available from Nisshin Oil Mills
*16 Absorption Refined Lanolin: Crodalan SWL available from Croda
*17 Lanolin Oil: Lanolin Oil available from Croda
*18 Trioctanoin: Hexalan available from Nisshin Oil Mills, Ltd.
*19 Isotridecyl Isononanoate: Crodamol TN available from Croda

Method of Preparation

The make-up compositions of Examples 9-13 are suitably prepared as follows: First, a mixture of component numbers 1 through 7 are heated to disperse at a speed of from 1500 rpm to 2500 rpm using a Homomixer at 90° C. in a sealed tank. Separately, a mixture of component numbers 8 through 11 are dissolved at 90° C. This solution (component numbers 8-11) is added to the dispersion (component numbers 1-7), and the mixture is further dispersed at a speed of from 500 rpm to 700 rpm using a Homomixer to form a hydrophobic gel. Next, component numbers 12 through 22, as present, are heated to dissolve at 80° C. in a sealed tank, followed by adding the above hydrophobic gel and component number 23, and the mixture is dispersed at 80° C. using a disper to make a lipophilic dispersion. The obtained dispersion is adjusted to a temperature of 80° C. Finally, the dispersion is filled in an air-tight container and allowed to cool to room temperature.

These embodiments represented by the previous examples are useful as skin care products. When applied to the facial skin, they provide many advantages. For example, they can provide improvement in the areas of skin tone, skin lightening, lightening of skin spots, skin sallowness reduction, and fine wrinkle reduction. Significant improvements in the benefits above are observed when the examples are used daily for a period of at least 4 weeks.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition comprising:
(1) from about 0.001% to about 10% of a flavonoid compound selected from the group consisting of hesperidin, glucosyl hesperidin, rutin, glucosyl rutin, glucosyl myricitrin, glucosyl isoquercitrin, glucosyl quercitrin, methyl hesperidin, and mixtures thereof;
(2) from about 0.01% to about 15% of a vitamin B3 compound; and
(3) a dermatologically acceptable oil continuous phase carrier.

2. The composition of claim 1 further comprising from 0.001% to about 15% of a vitamin B6 compound.

3. (canceled)

4. The composition of claim 1 wherein the flavonoid compound is glucosyl hesperidin.

5. The skin care composition of claim 1 wherein the oil continuous phase carrier is a water-in-oil emulsion comprising by weight of the entire composition:
(a) from about 20% to about 80% of an oil component;
(b) from about 0.1% to about 10% of a lipophilic surfactant having an HLB of less than about 8;
(c) from about 10% to about 60% of water;
(d) from 0% to about 30% of optional powder component; and
(e) from 0% to about 30% of optional thickener.

6. The skin care composition of claim 5 comprising from about 0,1% to about 30% of a silicone elastomer thickener.

7. The skin care composition of claim 1 wherein the oil continuous phase carrier comprises by weight of the entire composition:
(a) from about 20% to about 80% of an oil component;
(b) from about 0.1% to about 10% of a lipophilic surfactant having an HLB of less than about 8;
(c) from 0% to about 30% of optional powder component; and
(d) from 0% to about 30% of optional thickener.

8. A method of lightening the skin comprising the step of applying to the skin a composition comprising:
(1) from about 0.001% to about 10% of a flavonoid compound selected from the group consisting of hesperidin, glucosyl hesperidin, rutin, glucosyl rutin, glucosyl myricitrin, glucosyl isoquercitrin, glucosyl quercitrin, methyl hesperidin, and mixtures thereof;
(2) from about 0.01% to about 15% of a vitamin B3 compound; and
(3) a dermatologically acceptable oil continuous phase carrier.

9. A method of providing anti-aging benefit to the skin comprising the step of applying to the skin a composition comprising:
(1) from about 0.001% to about 10% of a flavonoid compound selected from the group consisting of hesperidin, glucosyl hesperidin, rutin, glucosyl rutin, glucosyl myricitrin, glucosyl isoquercitrin, glucosyl guercitrin, methyl hesperidin, and mixtures thereof:
(2) from about 0.01% to about 15% of a vitamin B3 compound; and
(3) a dermatologically acceptable oil continuous phase carrier.
